(19) United States
(12) Patent Application Publication
(10) Pub. No.: US 2013/0072937 A1
(43) Pub. Date: Mar. 21, 2013

(54) INSTRUMENTS FOR MINI-INVASIVE SPINAL COLUMN SURGERY AND USES THEREOF

(71) Applicant: Jerome Levieux, Geneve (CH)

(72) Inventor: Jerome Levieux, Geneve (CH)

(73) Assignee: SPINEART SA, Geneve (CH)

(21) Appl. No.: 13/699,749

(22) PCT Filed: Jun. 3, 2011
(86) PCT No.: PCT/FR2011/051267

§ 371 Date: Nov. 26, 2012

(30) Foreign Application Priority Data
Jun. 4, 2010 (FR) 1054383

(51) Int. Cl. A61B 17/16

(52) U.S. Cl. CPC A61B 17/1604
USPC 606/79

(57) ABSTRACT

A pedicle perforator (1) includes a handle (2) and a shaft (3) which ends opposite the handle (2) with an end (4) that has a perforated shape (hereinafter referred to as “tip”), characterized in that the shaft (3) includes a protruding centering device (7) for a cylindrical surface, provided in a region of the shaft (3) opposite the handle (2), a hollow guide tube (11) having an internal diameter of 4 to 20 mm, an external diameter of 5 to 22 mm and a length of 4 to 25 cm, including an end that has reliefs for attachment (12), for use together with the pedicle perforator, and a set including a pedicle perforator (1) and a hollow guide tube (11), wherein the pedicle perforator (1) can slide loosely in this hollow guide tube.

The present invention relates to instruments for minimally invasive spinal surgery and to uses thereof.

The many diseases of the spine are treated in a variety of ways, one of which is surgery. Depending on the indications, patients are offered different types of surgery. One of these is osteosynthesis. Osteosynthesis involves placing screws in the vertebral pedicles and connecting these screws by bars or plates in order to immobilize the diseased segment. One variant involves connecting the screws by flexible or semi-rigid bars (or systems) in order to stabilize the diseased segment without immobilizing it. In all these cases, the surgeon first places screws in the vertebral pedicles.

It is only recently that these techniques have been performed in a minimally invasive manner, that is to say with the surgeon making small incisions and fitting the screws in place through tubes (blind).

In minimally invasive surgery, the pedicle screws are fitted in place by way of tubes. The surgeon presently locates the entry of the pedicle using a system composed of a trocar and of a small needle called a “Jamshidi” after the inventor. When the entry of the pedicle has been located, the surgeon passes what is called a Kirschner wire or K-wire through the trocar and drives it into the pedicle under radiographic monitoring.

When the K-wire is in place, the surgeon passes a series of dilator tubes of increasing diameters around the K-wire in order to retract the muscles, and he then removes all of the tubes except the largest one. The pedicle screw and its implant holder are cannulated. They are then implanted by passing them over the K-wire. In this way, it is possible to ensure that the screw is correctly implanted in the pedicle, since it is guided by the K-wire fitted in the pedicle.

The advantage of this type of surgery is that the incision is smaller than in the conventional invasive technique (called open surgery) and leaves few scars. In addition, muscle disinsertion is almost zero. Overall, the patient has a better postoperative course, in particular reduced postoperative pain.

However, this technique is developing very slowly, since it is still more difficult to perform at present than the conventional invasive technique.

In addition, it has several disadvantages:

the implants and the instruments have to be cannulated, which increases their cost price;

the K-wire is disposable, which also increases costs;

the K-wire is a dangerous tool and must therefore be handled with care. For example, when implanting a screw, the procedure must be continuously monitored by X-ray in order to check that the K-wire is not driven by the screw.

Another disadvantage, therefore, is that this technique increases the radiation exposure of the patient and of the medical personnel.

It would be desirable for screws that are easy to produce and inexpensive, in particular not cannulated, to be placed safely in the vertebral pedicles by minimally invasive surgery.

For this, it would be desirable to have an instrument or a set of instruments that are simple, easy to produce and inexpensive.

It would also be desirable to allow the surgeon to place screws safely in the vertebral pedicles without the disadvantages associated with the existing systems, in particular by reducing the number of radiographic checks.

Since the K-wire is a dangerous instrument and must be handled with care, it would be desirable not to have to use it.

Now, after extensive research, the applicant has developed a novel system for minimally invasive surgery that is entirely satisfactory.

The invention overcomes the disadvantages of the prior art by using a large dilation tube, usually the last dilation tube or “guide tube” for guiding the screw into the pedicle. The position and orientation of this tube must be precise. It is for this reason that a pedicle perforator of the conventional type, such as those used in standard invasive surgery of the spine, has been conceived but modified so as to place and orient the guide tube optimally and, in the final analysis, so as to place and orient a pedicle screw optimally.

Moreover, the guide tube advantageously has teeth or tips or other types of fasteners, such that it is stable once in contact with the bone.

It is thus possible, in particular, to avoid using K-wires, cannulated screws and even “Jamshidi” trocars and, therefore, to reduce the costs and disadvantages of this surgery.

It is for this reason that the present application relates to a pedicle perforator comprising a handle and a shaft, the latter terminating opposite the handle with an end having a perforating shape (hereinafter called the “tip”), characterized in that the shaft comprises a protruding centering device for a cylindrical surface, provided in a region of the shaft opposite the handle. A pedicle perforator is commonly called a pedicle probe in English.

It should be noted that, in the present application, the indefinite article must conventionally be considered as a generic plural (meaning “at least one” or else “one or more”), except when the context shows the contrary (1 or “a single”). Thus, for example, when it is stated above that a protruding centering device is provided, this means provision of one or more protruding centering devices.

In the present application and in the text below, “a protruding centering device” is understood to mean a device that keeps the shaft in an axial position in a cylinder and at the same time is able to allow the shaft to slide in this cylinder. The shaft can slide in this cylinder with minimal friction, i.e. limited play is provided between the centering device and the cylinder to permit good centering. This centering device is fitted protruding from the shaft.

The centering device can be fine or solid. A fine centering device comprises, for example, rods or fins or a combination of a ring and rods or fins. A solid centering device comprises, for example, an elongate cylinder or an hourglass-shaped or shuttle-shaped elongate cylinder. It will be appreciated that the fact of being solid does not prevent it from being hollow.

A fine centering device comprises, for example, two or more sets of rods or fins spaced apart from one another. As regards a solid centering device, preferably only one will be provided.

For good centering, the centering device, when there is only one, is provided in a region of the shaft opposite the handle, i.e. near the tip. It is sufficiently long to ensure that the shaft and the tube are coaxial.

It is then provided in a region of the shaft at 1 cm to 25 cm from the end of the tip, preferably 2 cm to 20 cm from the end of the tip, in particular 3 cm to 15 cm from the end of the tip, very particularly 4 cm to 10 cm from the end of the tip.

Several centering devices will be provided in a region of the shaft at 1 cm to 25 cm from the end of the tip, preferably 2 cm to 20 cm from the end of the tip, in particular 3 cm to 15 cm from the end of the tip, very particularly 4 cm to 10 cm from the end of the tip.

It will be possible, for example, to provide 4, 3 and advantageously 2 centering devices.

A centering device will have a length of 20 mm to 250 mm, preferably 30 to 200 mm, in particular 50 to 150 mm, very particularly 60 to 100 mm. This length will be evaluated by the distance between the orthogonal projections, on the shaft, of the end tips of the centering device. For a cylinder, it will be the length of the cylinder.

In preferred conditions of implementation of the invention, the internal diameter of the above cylinder capable of allowing the shaft and its centering device to slide is approximately 14.5 mm, for example from 12 to 16 mm.

The shaft can have, for example, a length of 80 to 400 mm, preferably 100 to 300 mm, in particular 120 to 250 mm, very particularly 150 to 220 mm.

Preferably, the shaft will have a length of 100 to 300 mm with a centering device having a length of 3 cm to 20 cm, and in particular the shaft will have a length of 120 to 250 mm with a centering device having a length of 5 cm to 15 cm. In preferred conditions of use, these centering devices can slide in an above cylinder having an internal diameter of 12 to 16 mm.

In general, a shaft comprising a protruding centering device will have (except for the tip) an external diameter of 2 to 12 mm, preferably 3 to 10 mm, in particular 4 to 9 mm, very particularly 5 to 8 mm.

The shaft will usually be solid.

The protruding centering device will be a part that is attached to the shaft or that is formed in one piece with the shaft. Although the protruding centering device is preferably rigidly connected to the shaft, that is to say turns at the same time as the shaft, the protruding centering device can also turn separately from the shaft, which then serves as an axis of rotation for it.

The shaft of a pedicle perforator of the invention can be made of any material suitable for its use, for example of polymer alloyed with carbon, such as polymer alloyed with radiopaque materials, and can in particular be made of titanium or titanium alloy or preferably stainless steel.

The protruding centering devices of the invention can be made of any polymer material, such as alloyed polymer, and in particular of titanium or titanium alloy or preferably stainless steel.

All the features of a pedicle perforator of the invention as defined above can be combined with one another.

The present invention also relates to a method for producing pedicle perforators as have been defined above, characterized in that the shaft is machined or forged, the centering device is machined, and then a handle is fitted. According to another method, a centering device is attached to the shaft before or after a handle is fitted.

The pedicle perforators of the present invention can be used as follows:

a preliminary hole is formed with the aid of a squared point guided under X-ray monitoring,

dilator tubes of increasing diameters are engaged on this squared point, after which they are all removed except the largest tube, which is maintained in contact with the bone,

the pedicle perforator of the present invention is then inserted into the large tube. The tip of the pedicle perforator is thus guided safely to the preliminary hole; the perforator is then used conventionally to pierce the pedicle,

once the pedicle has been pierced exactly to a sufficient depth, the surgeon removes the pedicle perforator and keeps the tube in its position. He can perform this operation by hand or with the aid of an articulated immobilizing arm,

a pedicle screw is then inserted into the guide tube. The pedicle screw is guided by the guide tube, and it has a correct position and a correct orientation for entering the hole made by the pedicle perforator.

It will be noted that a pedicle screw has a screwing part with a length of several cm, in excess of 2.5 cm, often in excess of 3 cm, generally in excess of 4 cm, and possibly in excess of 5 cm. The features of the pedicle perforator take account of this constraint. For example, the end of the shaft having the perforating shape has an effective length, that is to say a length permitting perforation, with a length of several cm.

The conventional type of perforator being currently used is a very reliable tool for piercing a pedicle with precision.

To the knowledge of the applicant, the outer tube above is a novel product.

It is for this reason that the present application also relates to a hollow guide tube with an internal diameter of 4 to 20 mm, in particular 10 mm to 18 mm, and preferably about 14.5 mm, an external diameter of 5 to 22 mm, in particular 12 mm to 20 mm, preferably about 17 mm, and a length of 4 to 25 cm, in particular 8 cm to 18 cm, and preferably about 14 cm, having an end provided with fastening reliefs. The fastening reliefs can in particular be tips, teeth, etc. The fastening device can also be composed of long and fine screws for screwing the tube onto the bone.

Preferably, the hollow guide tube will have an internal diameter of 10 mm to 18 mm, for an external diameter of 12 mm to 20 mm, and a length of 8 cm to 18 cm.

The internal diameter of the hollow guide tube having an end provided with fastening reliefs is intended for guiding a pedicle perforator of the invention and then a pedicle screw.

A hollow guide tube of the invention can be made of any material suitable for its use, for example of polymer, polymer alloyed with carbon, polymers alloyed with radiopaque materials, and can in particular be made of stainless steel or preferably titanium or titanium alloy.

The present invention also relates to a kit for pedicle perforation and placement of pedicle screws in minimally invasive spinal surgery, comprising at least one pedicle perforator as defined above and at least one hollow guide tube as defined above, the design of the perforator and the internal diameter of the hollow guide tube allowing the pedicle perforator to slide with minimal friction in the hollow guide tube.

The pedicle perforators of the present invention have very advantageous properties and qualities.

In particular, by virtue of their guide region, they allow the guide tube to be placed and oriented in an optimal manner. This then allows the guide tube, maintained in its position, to safely guide a screw into the vertebral pedicle. Perforators (without protruding centering device) are much used since they are very effective in targeting the pedicle. For this reason, the perforators of the present invention (with protruding centering device) allow surgeons to perform minimally invasive spinal surgery using a tool with which they are familiar and avoiding the disadvantages associated with the use of K-wires. They can be produced easily and quickly, and the cost of producing them is therefore low.

The surgeon can perform minimally invasive spinal surgery by operating using a technique similar to the technique referred to as open surgery. This therefore also has the advantage of opening up the possibility of minimally invasive spinal surgery to all surgeons. Moreover, by virtue of this technique, the number of X-ray checks is less than that conventionally carried out during minimally invasive spinal surgery requiring the use of K-wires.

Moreover, this device is particularly adapted to the minimally invasive techniques.

These qualities are illustrated below. They justify the use of the above-described pedicle perforators for fitting vertebral pedicle screws according to the minimally invasive technique and in particular in minimally invasive osteosynthesis of the thoracolumbar vertebrae.

It is for this reason that the present invention also relates to a method for fitting vertebral pedicle screws, in which method

a preliminary hole is formed in a vertebral pedicle with the aid of a point,

dilator tubes of increasing diameters are engaged on this point, after which they are all removed except the largest tube (guide tube), which is maintained in contact with the bone,

a pedicle perforator of the present invention is inserted into the guide tube in order to pierce the pedicle,

the pedicle perforator is removed and the guide tube is maintained in its position,

the pedicle screw, which is guided by the guide tube, is then inserted into the tube and then into the hole formed by the pedicle perforator.

Since the above devices are for surgical use, the present invention also relates to said devices when sterile, in particular packed in packaging that keeps them sterile.

The preferred conditions of use of the above-described pedicle perforators also apply to the other subjects of the invention that have been defined above, in particular to the methods for using them and producing them.

The invention will be better understood by reference to the attached drawings, in which

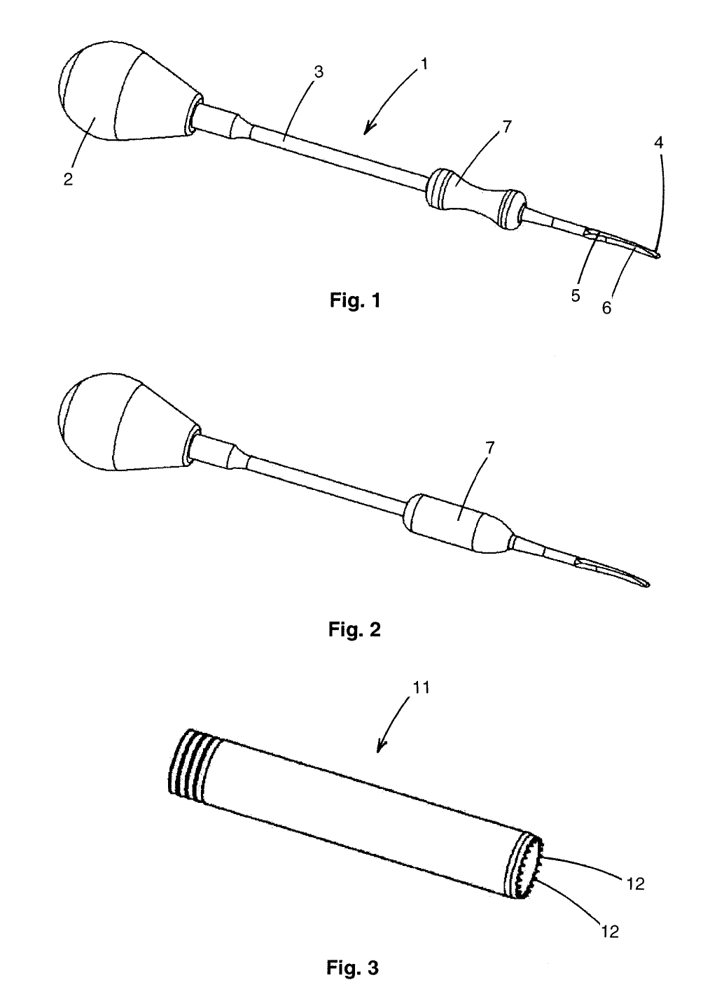

FIG. 1 shows a perspective view of a pedicle perforator of the present invention,

FIG. 2 shows a perspective view of a variant of the pedicle perforator of the present invention,

FIG. 3 shows a perspective view of a guide tube of the present invention,

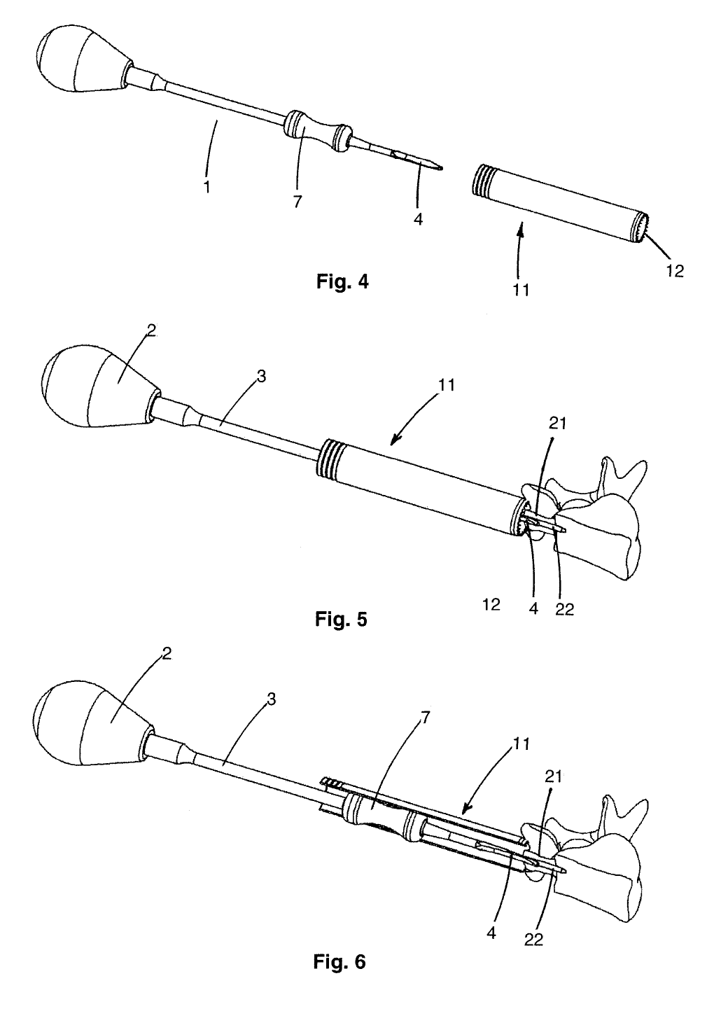

FIG. 4 shows a perspective view of a kit composed of a pedicle perforator and of a guide tube of the present invention,

FIG. 5 shows a use of a kit composed of a pedicle perforator and of a guide tube of the present invention, for piercing a vertebral pedicle,

FIG. 6 is a partially exploded view of FIG. 5.

FIG. 1, which is a perspective view of a pedicle perforator 1 of the present invention, shows the pear-shaped handle 2 with which it is possible to take hold of the pedicle perforator 1 and press it while applying, for example, a reciprocating rotation movement in order to perforate a pedicle. The handle 2 is rigidly connected to a shaft 3, which terminates opposite the handle 2 with an end 4 having a perforating shape, in this case comprising facets 5, 6. The shaft 3 comprises one centering device 7 for a cylindrical surface, provided in a region of the shaft 3 opposite the handle 2. The centering device 7 is in this case solid and hourglass-shaped and only one is provided.

The centering device 7 keeps the shaft 3 in an axial position in a cylinder and at the same time allows the shaft 3 to slide in this cylinder, as will be seen hereinafter. This centering device 7 is fitted protruding from the shaft 3, since its diameters are greater than the diameter of the shaft 3 at the location where it is fixed. The shaft 3 is centered in the centering device 7. The centering device 7 here is solid and hourglass-shaped and only one is provided.

The centering device 7, in this embodiment, is rigidly connected to the shaft 3.

It is provided in a region of the shaft 3 opposite the handle 2, i.e. near the tip 4. It is sufficiently long to ensure that the shaft 3 and the centering device 7 are coaxial.

In this illustrative embodiment, it has a length of 5 cm and is provided in the region of the shaft 3 at 3 cm to 8 cm from the end 4 with a perforating shape. Its maximum diameter is approximately 14.2 mm and its minimum diameter toward the middle of its length is approximately 14 mm.

The shaft 3 here has a length of approximately 200 mm measured between the end 4 and the opposite end at the face of the handle 2. The shaft 3 is solid.

Along most of its length, the shaft 3 has a constant external diameter of approximately 6 mm. Near its channel of introduction into the handle 2, the shaft 3 has an increased external diameter.

FIG. 2 shows a centering device 7 for a cylindrical surface, provided in a region of the shaft 3 opposite the handle 2. A single centering device 7 is provided here, and it is solid and basically of cylindrical shape, tapering toward the end 4 having a perforating shape.

FIG. 3 shows a guide tube 11. The guide tube 11 has an internal diameter of approximately 14.5 mm and an external diameter of approximately 17.5 mm. The centering device 7 is thus able to slide with minimal friction in the tube in order to guide the pedicle perforator 1 so that the latter perforates a pedicle exactly at the desired location. One of the ends of the tube 11 is provided with fastening reliefs, which here are teeth 12.

FIG. 4 shows a pedicle perforator 1 in the position when introduced into a guide tube 11. This dilator guide tube 11 is the outer guide tube left in place after installation of the dilator tubes of increasing diameters.

FIG. 5 shows a kit composed of a pedicle perforator 1 and of a guide tube 11 of the present invention, in action for piercing a vertebral pedicle 21.

A pedicle perforator 1 has been introduced into a guide tube 11. Guided by the centering device 7 (visible in the following figure), the end 4 having a perforating shape is centered in the guide tube 11. The latter, by virtue of its ends provided with fastening reliefs, has been correctly placed so as to target the site to be pierced without slipping. The end 4 having a perforating shape has been activated in rotation by manipulation of the handle 2 and has pierced the vertebral pedicle 21 at the site and at the angle chosen in order to create a channel 22 in the vertebral pedicle 21. After removal of the pedicle perforator 1, and with the guide tube 11 being maintained in place by virtue of its ends provided with fastening reliefs, a pedicle screw with a suitable head diameter (approximately 14 mm) can be guided safely toward the channel 22 forming a preliminary hole in order to be screwed.

FIG. 6 shows the same elements as in FIG. 5, and other elements too, since the figure is an exploded view.

The same principle has also been followed to produce five pedicle perforators with the following dimensions and type:

| | 2 | 3 | 4 | 5 | 6 |
|---|---|---|---|---|---|
| Material used | Stainless | Titanium | Radel | Carbon | Polymer |
| | steel | | | polymer | |
| | | | | composite | |
| Length of shaft | 18.0 | 20 | 15 | 18 | 15 |
| in cm, | | | | | |
| excluding | | | | | |
| handle | | | | | |
| External | 5 | 5.5 | 6 | 5 | 7 |
| diameter of | | | | | |
| shaft in mm | | | | | |
| Number of | 1 | 2 | Two | 3 | 3 |
| centering | | | regions | | |
| devices | | | 9 mm in | | |
| | | | length | | |
| Nature of | Radel | Titanium | Radel | Carbon | Polymer |
| centering | | | | polymer | |
| device | | | | composite | |
| Shape of | Cylinder | Cylinder | Hourglass | Cylinder | 6 Fins |
| centering | | | | | |
| device | | | | | |

1. A pedicle perforator (1) comprising a handle (2) and a shaft (3), the latter terminating opposite the handle (2) with an end (4) having a perforating shape (hereinafter called the “tip”), characterized in that the shaft (3) comprises a protruding centering device (7) for a cylindrical surface, provided in a region of the shaft (3) opposite the handle (2).

2. The pedicle perforator as claimed in claim 1, characterized in that the centering device (7) is solid like an elongate cylinder or an hourglass-shaped or shuttle-shaped elongate cylinder.

3. The pedicle perforator as claimed in claim 1, characterized in that there is a single centering device (7) on the shaft (3).

4. The pedicle perforator as claimed in claim 3, characterized in that the single centering device (7) is provided in a region of the shaft at 2 cm to 20 cm from the end of the tip.

5. The pedicle perforator as claimed in claim 1, characterized in that the centering device (7) has a length of 50 to 150 mm.

6. The pedicle perforator as claimed in claim 1, which can be used with a hollow cylindrical guide tube, characterized in that the dimensions of a centering device (7) are such that the internal diameter of a cylinder (11) capable of allowing the shaft (3) and its centering device (7) to slide is from 12 to 16 mm.

7. The pedicle perforator as claimed in claim 1, characterized in that the shaft (3) has a length of 100 to 300 mm.

8. A hollow guide tube (11) with an internal diameter of 4 to 20 mm, an external diameter of 5 to 22 mm and a length of 4 to 25 cm, for use together with a pedicle perforator (1) as defined in claim 1, having an end provided with fastening reliefs (12).

9. A kit comprising a pedicle perforator (1) as defined in claim 1 and a hollow guide tube (11) having an internal diameter of 4 to 20 mm, an external diameter of 5 to 22 mm, a length of 4 to 25 cm, and an end provided with fastening reliefs (12), the pedicle perforator (1) being able to slide with minimal friction in this hollow guide tube.

10. The pedicle perforator as claimed in claim 2, characterized in that there is a single centering device (7) on the shaft (3).
